(19) United States
(12) Patent Application Publication
(10) Pub. No.: US 2022/0409265 A1
(43) Pub. Date: Dec. 29, 2022

(54) ANTI-BACKDRIVE ASSEMBLY FOR VESSEL SEALING INSTRUMENT

(71) Applicant: Covidien LP, Mansfield, MA (US)

(72) Inventors: Grant T. Sims, Boulder, CO (US); William R. Whitney, Boulder, CO (US); Jason G. Weihe, Longmont, CO (US); James D. Allen, IV, Broomfield, CO (US); Jason T. Sanders, Longmont, CO (US); Alyssa M. Sawyer, Broomfield, CO (US); Curtis M. Siebenaller, Frederick, CO (US)

(21) Appl. No.: 17/849,032

(22) Filed: Jun. 24, 2022

Related U.S. Application Data
(60) Provisional application No. 63/214,937, filed on Jun. 25, 2021.

(51) Int. Cl. A61B 18/14; A61B 18/12

(52) U.S. Cl. CPC A61B 18/1447; A61B 18/1206; A61B 2017/2936

(57) ABSTRACT

A vessel sealing instrument includes a housing having a shaft extending from a distal end thereof having an end effector assembly including a pair of opposing first and second jaw members operably coupled thereto. A drive assembly is disposed within the housing and is configured to move the jaw members upon actuation thereof between an open position and a closed position for clamping tissue with a closure pressure within the range of about 3 kg/cm² to about 16 kg/cm². An anti-backdrive assembly is operably disposed within the housing and includes a drive wedge. A solenoid controller is operably coupled to the drive wedge and is configured to selectively move the drive wedge into the drive assembly upon activation thereof to increase the closure pressure between the jaw members in response to tissue expansion during sealing.

### CROSS REFERENCE TO RELATED APPLICATION

This application claims the benefit of U.S. Provisional Application Ser. No. 63/214,937 filed Jun. 25, 2021, the entire contents of which being incorporated by reference herein.

### FIELD

The present disclosure relates to surgical instruments and, more particularly, to anti-backdrive assemblies for vessel sealing instruments configured to maintain closure pressure during sealing.

### BACKGROUND

A surgical forceps is a pliers-like surgical instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is grasped under a closure pressure suitable to seal vessels or tissue, the actuation mechanism (e.g., handle) is locked during the delivery of electrosurgical energy to produce a seal. In some instance the surgeon holds the actuation mechanism during electrosurgical activation. During sealing, the tissue naturally expands against the closure pressure which, in some instances, can affect the resulting tissue seal as the closure pressure no longer falls within a particular closure pressure range.

Accordingly, there exists a need to maintain the closure pressure within the desired closure pressure range during the sealing process.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a vessel sealing instrument includes a housing having a shaft extending from a distal end thereof having an end effector assembly including a pair of opposing first and second jaw members operably coupled thereto. A drive assembly is disposed within the housing and is configured to move the jaw members upon actuation thereof between an open position and a closed position for clamping tissue with a closure pressure within the range of about 3 kg/cm² to about 16 kg/cm². An anti-backdrive assembly is operably disposed within the housing and includes a drive wedge. A solenoid controller is operably coupled to the drive wedge and is configured to selectively move the drive wedge into the drive assembly upon activation thereof to increase the closure pressure between the jaw members in response to tissue expansion during sealing.

In aspects according to the present disclosure, the solenoid controller selectively moves the drive wedge in response to a sealing algorithm. In other aspects according to the present disclosure, the solenoid controller selectively moves the drive wedge in response to a sensor.

In aspects according to the present disclosure, the housing includes a moveable handle disposed therein operably coupled to the drive assembly, actuation of the moveable handle correspondingly moves the drive assembly to move the jaw members between open and closed positions. In other aspects according to the present disclosure, a portion of the handle is disposed between a drive ring and a biasing flange of the drive assembly, actuation of the handle biases the biasing flange and the drive ring relative to one another to move the jaw members. In still other aspects according to the present disclosure, the drive wedge is disposed between the handle and the drive ring or the handle and the biasing flange such that upon actuation of the solenoid controller, the drive wedge further biases the drive ring and the biasing flange relative to one another.

In aspects according to the present disclosure, the drive assembly includes a spring configured to regulate the closure pressure between the jaw members.

Provided in accordance with aspects of the present disclosure is a vessel sealing instrument including a housing having a shaft extending from a distal end thereof including an end effector assembly having a pair of opposing first and second jaw members operably coupled thereto. A drive assembly is disposed within the housing and is configured to move the jaw members upon actuation thereof between an open position and a closed position for clamping tissue with a closure pressure within the range of about 3 kg/cm² to about 16 kg/cm². An anti-backdrive assembly is operably disposed within the housing and includes a drive wedge. A thermally controlled spring is operably coupled to the drive wedge and is transitional between a first configuration and one or more second configurations upon activation thereof via a change in temperature. The thermally controlled spring is configured to selectively move the drive wedge into the drive assembly upon transition thereof to increase the closure pressure between the jaw members in response to tissue expansion during sealing.

In aspects according to the present disclosure, the thermally controlled spring is operably coupled to a thermal controller configured to selectively transition the thermally controlled spring. In other aspects according to the present disclosure, the thermal controller selectively moves the thermally controlled spring and drive wedge in response to a sealing algorithm. In yet other aspects according to the present disclosure, the thermal controller selectively moves the thermally controlled spring and drive wedge in response to a sensor.

In aspects according to the present disclosure, the housing includes a moveable handle disposed therein operably coupled to the drive assembly, actuation of the moveable handle correspondingly moves the drive assembly to move the jaw members between open and closed positions. In other aspects according to the present disclosure, a portion of the handle is disposed between a drive ring and a biasing flange of the drive assembly, actuation of the handle biases the biasing flange and the drive ring relative to one another to move the jaw members. In yet other aspects according to the present disclosure, the drive wedge is disposed between the handle and the drive ring or the handle and the biasing flange such that upon actuation of the thermal controller and thermally controlled spring, the drive wedge further biases the drive ring and the biasing flange relative to one another.

In aspects according to the present disclosure, the drive assembly includes a spring configured to regulate the closure pressure between the jaw members.

In aspects according to the present disclosure, the drive wedge and thermally controlled spring are disposed within a cutout defined within the handle.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1A is a perspective view of an electrosurgical forceps provided in accordance with the present disclosure having in-line electrosurgical activation;

FIG. 1B is a perspective view of an electrosurgical forceps provided in accordance with another embodiment of the present disclosure having a ratchet-like handle assembly;

FIG. 2A is an enlarged, perspective view of an end effector assembly of the electrosurgical forceps of FIG. 1 wherein first and second jaw members of the end effector assembly are disposed in a spaced-apart position;

FIG. 2B is an enlarged, perspective view of the end effector assembly of FIG. 2A wherein the first and second jaw members are disposed in an approximated position;

FIG. 3A is a side view of a proximal portion of the electrosurgical forceps of FIG. 1 with a movable handle and trigger thereof disposed in respective un-actuated positions;

FIG. 3B is a side view of the proximal portion of the electrosurgical forceps shown in FIG. 3A with the movable handle disposed in an actuated position and the trigger disposed in the un-actuated position;

FIG. 3C is a side view of the proximal portion of the electrosurgical forceps shown in FIG. 3A with the movable handle and trigger disposed in respective actuated positions;

FIG. 4A is a side view of another proximal portion of the electrosurgical forceps of FIG. 1 with portions removed to illustrate a trigger assembly thereof with the trigger disposed in the un-actuated position;

FIG. 4B is a side view of the proximal portion of the electrosurgical forceps shown in FIG. 4A with portions removed to illustrate the trigger assembly with the trigger disposed in the actuated position;

FIGS. 5A-5B are internal side views of a drive assembly of the electrosurgical forceps;

FIG. 6 is an enlarged, schematic side view of the drive assembly of FIG. 5A;

FIG. 7A is an enlarged, schematic side view of one embodiment of an anti-backdrive assembly for use with the drive assembly in accordance with the present disclosure;

FIG. 7B is an enlarged, schematic side view of another embodiment of an anti-backdrive assembly for use with the drive assembly in accordance with the present disclosure; and

FIG. 8 is an enlarged, schematic side view of one embodiment of an anti-backdrive assembly for use with a drive sleeve of the electrosurgical forceps in accordance with the present disclosure;

### DETAILED DESCRIPTION

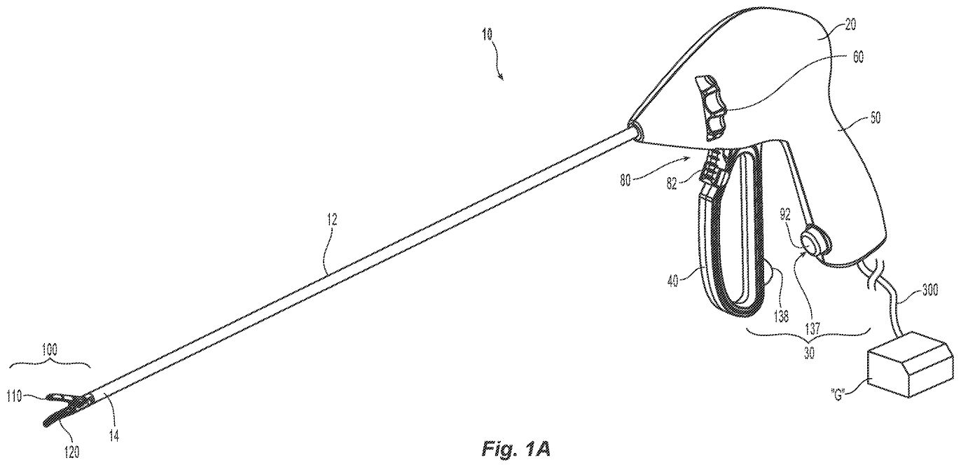

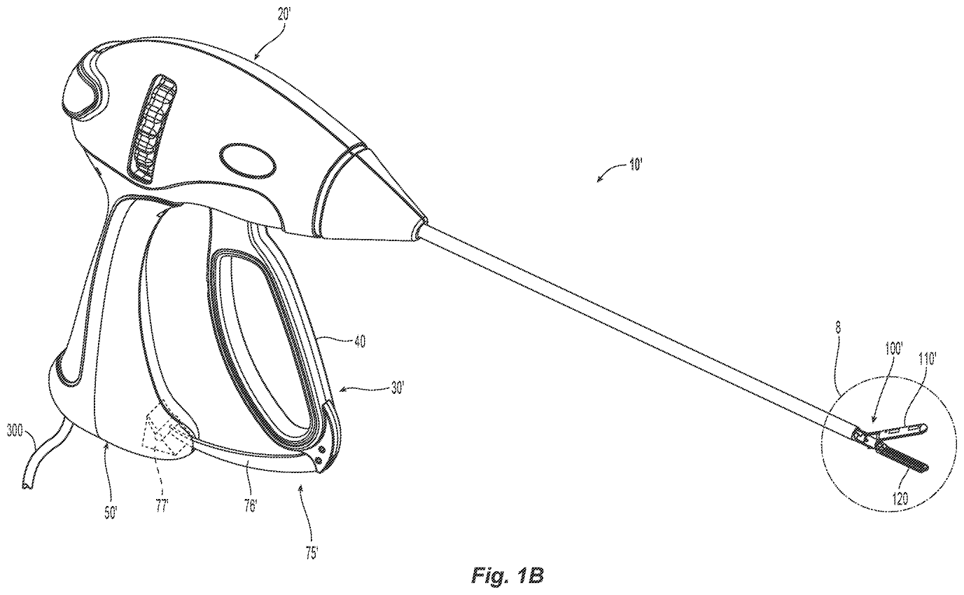

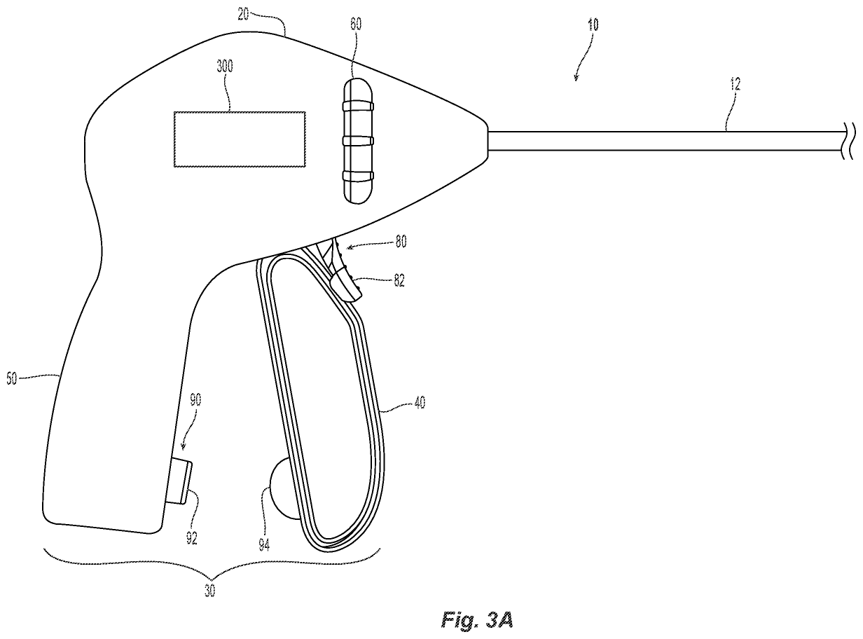

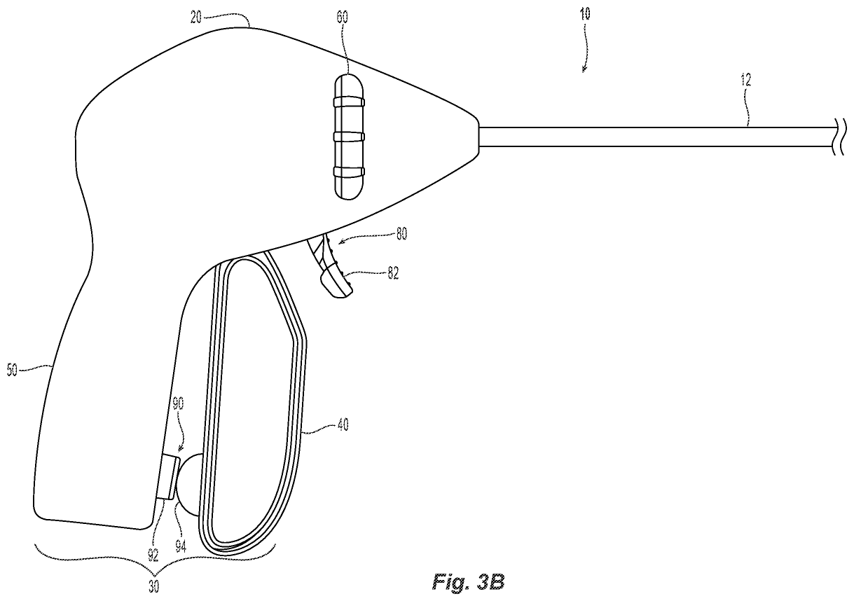

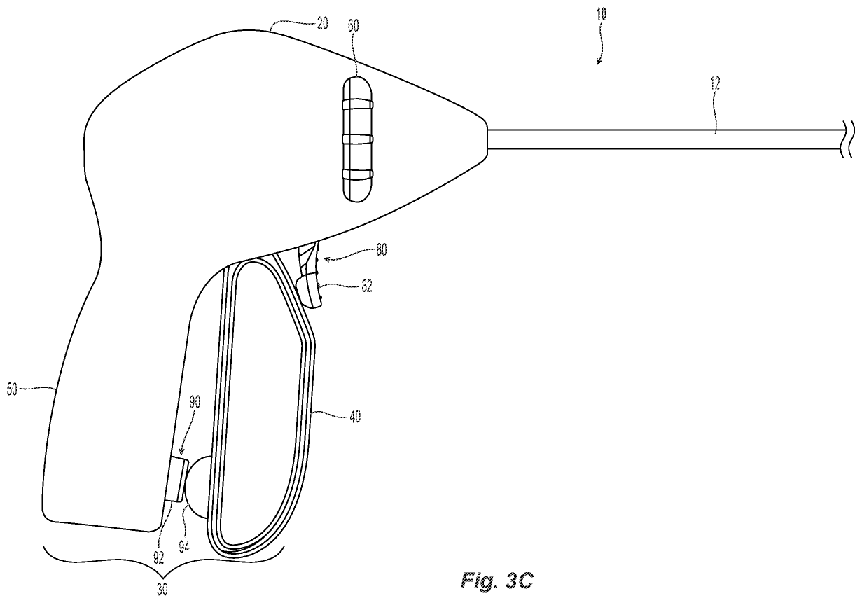

Referring to FIG. 1A, a surgical instrument provided in accordance with the present disclosure is shown configured as a bipolar electrosurgical forceps 10 for use in connection with endoscopic surgical procedures, although the present disclosure may be equally applicable for use with other surgical instruments such as those for use in endoscopic and/or traditional open surgical procedures. Forceps 10 generally includes a housing 20, a handle assembly 30, a rotating assembly 60, a trigger assembly 80, an activation assembly 90 (FIGS. 3A-3C), and an end effector assembly 100 including first and second jaw members 110, 120.

Forceps 10 further includes a shaft 12 having a distal end portion 14 configured to engage (directly or indirectly) end effector assembly 100 and a proximal end portion 16 that engages (directly or indirectly) housing 20. Rotating assembly 60 is rotatable in either direction to rotate shaft 12 and end effector assembly 100 relative to housing 20 in either direction. Housing 20 houses the internal working components of forceps 10.

An electrosurgical cable 300 connects forceps 10 to an electrosurgical generator “G” or other suitable energy source, although forceps 10 may alternatively be configured as a handheld instrument incorporating energy-generating and/or power components thereon or therein. Cable 300 includes wires (not shown) extending therethrough, into housing 20, and through shaft 12, to ultimately connect electrosurgical generator “G” to jaw member 110 and/or jaw member 120 of end effector assembly 100. Activation button 92 of activation assembly 90 is disposed on housing 20 are electrically coupled between end effector assembly 100 and cable 300 to enable the selective supply of energy to jaw member 110 and/or jaw member 120, e.g., upon activation of activation button 92. However, other suitable electrical connections and/or configurations for supplying electrosurgical energy to jaw member 110 and/or jaw member 120 may alternatively be provided, as may other suitable forms of energy, e.g., ultrasonic energy, microwave energy, light energy, thermal energy, etc.

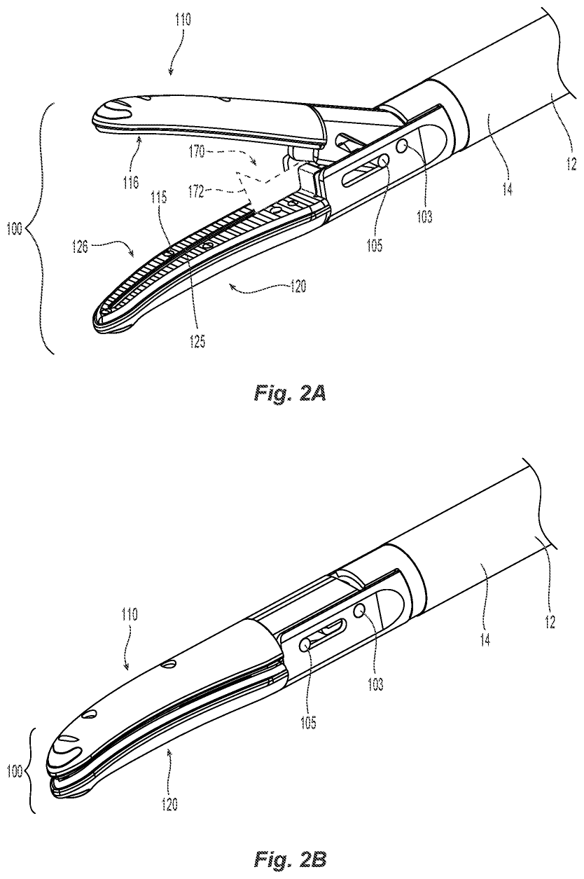

Forceps 10 additionally includes a knife assembly 170 (FIG. 2A) operably coupled to trigger assembly 80 and extending through housing 20 and shaft 12. One or both of jaw members 110, 120 defines a knife channel 125 (FIG. 2A) configured to permit reciprocation of a knife blade 172 (FIG. 2A) of knife assembly 170 (FIG. 2A) therethrough, e.g., in response to actuation of trigger 82 of trigger assembly 80. Trigger assembly 80 is described in greater detail below as are other embodiments of trigger assemblies configured for use with forceps 10.

With additional reference to FIGS. 2A and 2B, end effector assembly 100, as noted above, is disposed at distal end portion 14 of shaft 12 and includes a pair of jaw members 110 and 120 pivotable between a spaced-apart position and an approximated position for grasping tissue therebetween. End effector assembly 100 is designed as a unilateral assembly, e.g., wherein one of the jaw members 120 is fixed relative to shaft 12 and the other jaw member 110 is movable relative to both shaft 12 and the fixed jaw member 120. However, end effector assembly 100 may alternatively be configured as a bilateral assembly, e.g., wherein both jaw member 110 and jaw member 120 are movable relative to one another and with respect to shaft 12.

Each jaw member 110, 120 of end effector assembly 100 includes an electrically-conductive tissue-contacting surface 116, 126. Tissue-contacting surfaces 116 are positioned to oppose one another for grasping and treating tissue. More specifically, tissue-contacting surfaces 116, 126 are electrically coupled to the generator “G,” e.g., via cable 300, and activation button 92 to enable the selective supply of energy thereto for conduction through tissue grasped therebetween, e.g., upon activation of activation button 92. One or both of tissue-contacting surfaces 116, 126 may include one or more stop members 115 extending therefrom to define a minimum gap distance between electrically-conductive tissue-contacting surfaces 116, 126 in the approximated position of jaw members 110, 120, facilitate grasping of tissue, and/or inhibit shorting between electrically-conductive tissue-contacting surfaces 116, 126.

The stop member(s) 115 may be formed at least partially from an electrically-insulative material or may be effectively insulative by electrically isolating the stop member(s) from one or both of the electrically-conductive tissue-contacting surfaces 116, 126. The one or more stop members 115 may be disposed on one or both jaw members 110, 120 or on the tissue-contacting surfaces 116, 126 and are configured to regulate the distance therebetween. Details relating to various stop member designs are disclosed in U.S. Pat. Nos. 7,857,812, 10,687,887 the entire contents of each of which being incorporated by reference here.

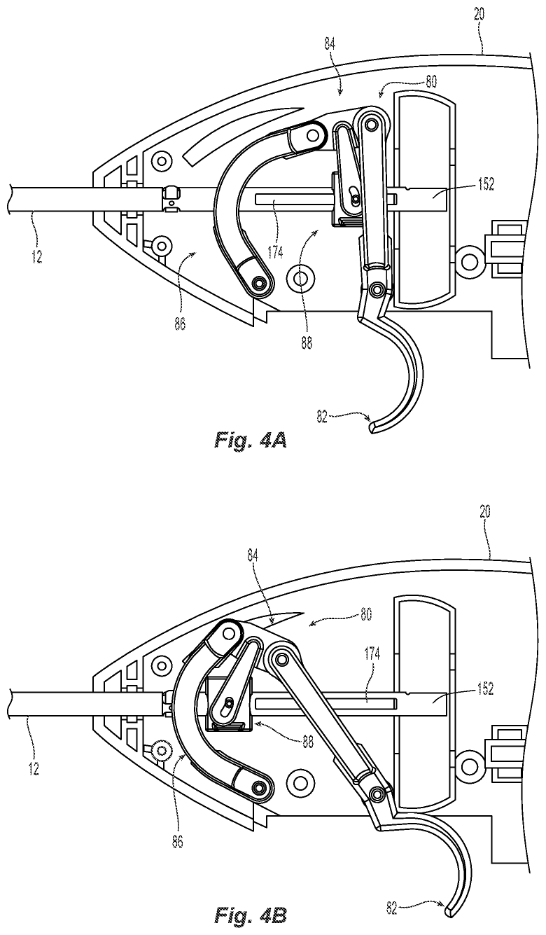

A pivot pin 103 of end effector assembly 100 extends transversely through aligned apertures defined within jaw members 110, 120 and shaft 12 to pivotably couple jaw member 110 to jaw member 120 and shaft 12. A cam pin 105 of end effector assembly 100 extends transversely through cam slots defined within jaw members 110, 120 and is operably engaged with a distal end portion of a drive bar 152 (FIGS. 4A and 4B) of a drive assembly 300 (only drive bar 152 (FIGS. 4A and 4B) of the drive assembly 300 is shown and drive assembly is generically represent by component 300 in FIG. 3A) such that longitudinal translation of drive bar 152 (FIGS. 4A and 4B) through shaft 12 translates cam pin 105 relative to jaw members 110, 120. Various drive assemblies are shown and described with respect to commonly-owned U.S. Pat. Nos. 7,857,812, 8,540,711, 7,384,420, 7,090,673, 7,101,372, 7,255,697, 7,101,371, 7,131,971, 7,083,618, and 10,842,553, the entire contents of each of which being incorporated by reference herein.

More specifically, distal translation of cam pin 105 relative to jaw members 110, 120 urges cam pin 105 distally through the cam slots to thereby pivot jaw members 110, 120 from the spaced-apart position towards the approximated position, although cam slots may alternatively be configured such that proximal translation of cam pin 105 pivots jaw members 110, 120 from the spaced-apart position towards the approximated position. One suitable drive assembly is described in greater detail, for example, in U.S. Pat. No. 9,655,673, the entire contents of which are hereby incorporated herein by reference.

Referring to FIGS. 1A-3C, handle assembly 30 includes a fixed handle 50 and an actuator, e.g., movable handle 40. Fixed handle 50 is integrally associated with housing 20 and movable handle 40 is movable relative to fixed handle 50. Movable handle 40 is ultimately connected to the drive assembly (not shown) that, together, mechanically cooperate to impart movement of jaw members 110 and 120 between the spaced-apart and approximated positions to grasp tissue between electrically-conductive surfaces 116, 126, respectively. More specifically, pivoting of movable handle 40 relative to fixed handle 50 from an un-actuated position towards an actuated position pivots jaw members 110, 120 from the spaced-apart position towards the approximated position. On the other hand, when movable handle 40 is released or returned towards the initial position relative to fixed handle 50, jaw members 110, 120 are returned towards the spaced-apart position.

A biasing spring (not shown) associated with movable handle 40 and/or the drive assembly may be provided to bias jaw members 110, 120 towards a desired position, e.g., the spaced-apart position or the approximated position. Various drive assemblies are shown and described in any one of the above-identified commonly-owned U.S. Patents referenced herein.

Fixed handle 50 operably supports activation button 92 of activation assembly 90 thereon in an in-line position, wherein activation button 92 is disposed in the actuation path of movable handle 40. In this manner, upon pivoting of movable handle 40 relative to fixed handle 50 from the actuated position to an activated position, protrusion 94 of movable handle 40 is urged into contact with activation button 92 to thereby activate activation button 92 and initiate the supply of energy to electrically-conductive surfaces 116, 126, e.g., to treat tissue grasped therebetween. Alternatively, actuation button 92 may be disposed in any other suitable position, on housing 20 or remote therefrom, to facilitate manual activation by a user to initiate the supply of energy to electrically-conductive surfaces 116, 126.

With reference to FIGS. 1A-2B and 4A-4B, as noted above, trigger assembly 80 is operably coupled to knife blade 172 of knife assembly 170. More specifically, trigger 82 of trigger assembly 80 is selectively actuatable, e.g., from an un-actuated position (FIGS. 3A and 4A) to an actuated position (FIGS. 3C and 4B), to deploy knife blade 172 distally through jaw members 110, 120 to cut tissue grasped between electrically-conductive surfaces 116, 126. Knife assembly 170 includes knife blade 172 and a knife bar 174 engaged with and extending proximally from knife blade 172 through shaft 12 and drive bar 152 into housing 20 where knife bar 174 is operably coupled with trigger assembly 80, as detailed below.

Referring to FIGS. 4A and 4B, trigger assembly 80 includes trigger 82, a link 84, e.g., a T-link 84, a link 86, e.g., an arcuate linkage 86 although other configurations, e.g., linear, angled, etc. are also contemplated, and a slider block 88. In this manner, trigger assembly 80 defines a four-bar mechanical linkage assembly for driving slider block 88 to actuate the knife blade 172. This and other types of trigger mechanism are also contemplated such as, for example, trigger mechanisms described in any one of the above-identified commonly-owned U.S. Patents referenced herein or U.S. patent application Ser. No. 16/558,477, the entire contents of each of which being incorporated by reference herein.

As mentioned above, pivoting of movable handle 40 relative to fixed handle 50 from an un-actuated position towards an actuated position pivots jaw members 110, 120 from the spaced-apart position towards the approximated position for grasping tissue therebetween. When fully grasped, the drive assembly 300 is configured to initially generate a closure pressure suitable for sealing vessels upon activation of electrosurgical energy from generator “G”. Maintaining closure pressures within the range of about 3 Kg/cm² to about 16 Kg/cm² are known to promote quality seals.

With in-line actuation instruments, the surgeon is typically required to maintain the handle 40 in position to continually maintain the closure pressure. For example and as shown in FIG. 1A, handle 40 is initially actuated under a light pressure to grasp and manipulate tissue prior to sealing as the jaw members 110, 120 may be closed without fully actuating handle 40 relative to handle 50. Once the tissue is properly positioned between jaw members 110, 120, the handle 40 may be fully actuated to close the jaw members 110, 120 about tissue within the above-noted pressure range and simultaneously activate the forceps 10 for sealing. With this type of forceps 10, the surgeon must maintain the handle 40 fully actuated to maintain the initial closure pressure. This is known as in-line activation.

Other forceps e.g., forceps 10′ of FIG. 1B include handle assemblies 30′ (including moveable handle 40′ and fixed handle 50′) that have a ratchet-like locking system 75′ affixed to a portion of the housing 20′ or handle assembly 30′ which is configured to lock handle 40′ relative to the fixed handle 50′ to initially generate and maintain the appropriate closure pressures between jaw members 110′, 120′ when locked. Ratchet-like locking system 75′ includes a flange 76′ extending from handle 40′ configured to mechanically engage and lock within a corresponding ramp 77′ (shown in phantom) disposed within handle 50′. Various such forceps and handle assemblies are shown in any one of the above-identified commonly-owned U.S. Patents referenced herein.

After the initial closure pressure within the above-identified range is generated and the jaw members 110, 120 (or 110′, 120′) are clamped on a vessel or on tissue, the forceps 10 (10′) is ready for activation. As mentioned above, during sealing the vessel or tissue expands against the jaw members, e.g., jaw members 110′, 120′, which may reduce the actual closure pressure during formation of the seal. If the closure pressure falls outside of the above-noted range, the seal may not be as effective.

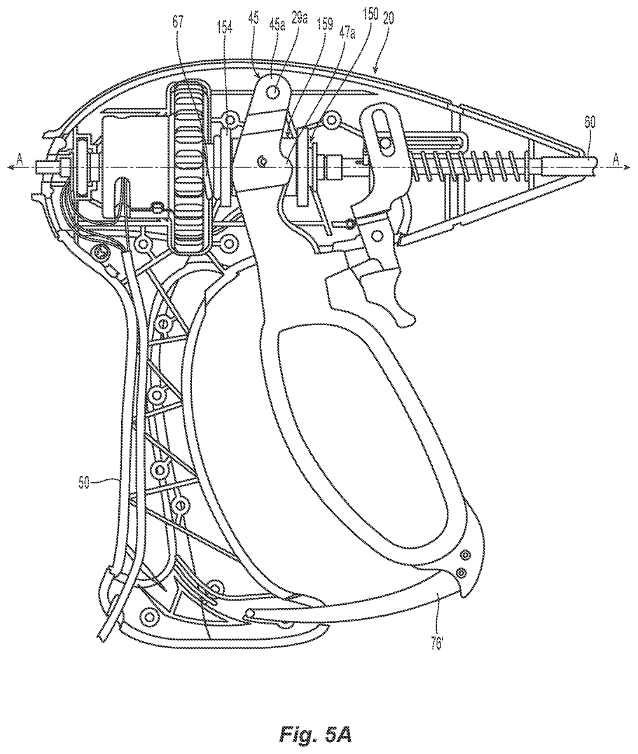

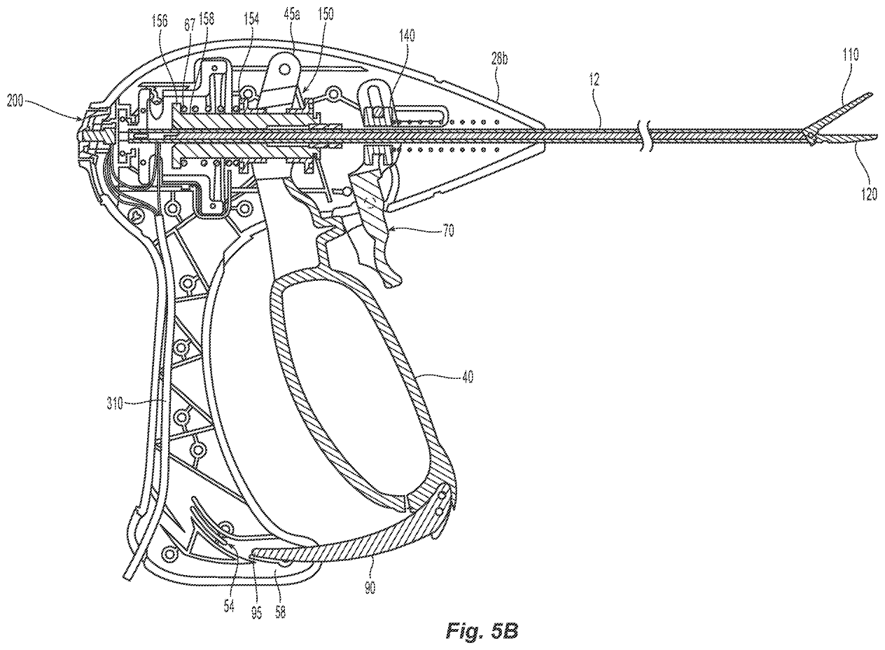

FIGS. 5A-8 show various drive assemblies which include one or more so-called “anti-backdrive assemblies” configured to maintain the required closure pressure or increase the sealing pressure as needed during the sealing process. All of the below-described anti-backdrive assemblies are configured to provide additional pressure to the jaw members, e.g., jaw members 110, 120, to offset the forces attributed to tissue expansion. It is envisioned that any of the below-described anti-backdrive assemblies may be: passive, e.g., prevent the jaw members from 110, 120 from moving during tissue expansion; proactive, e.g., anticipate tissue expansion and counteract the same; or reactive, e.g., measure the expansion or rate of expansion (feedback) and counteract the same.

Turning now to FIGS. 5A-5B which show a typical drive assembly for a surgical forceps generally identified as drive assembly 150. As mentioned above, movable handle 40 is selectively moveable from a first position relative to fixed handle 50 to a second position in closer proximity to the fixed handle 50 which, as explained below, imparts movement of the jaw members 110 and 120 relative to one another. The movable handle include a clevis 45 which forms a pair of upper flanges (flange 45a is shown) each having an aperture at an upper end thereof for receiving pivot pin 29a therethrough and mounting the upper end of the handle 40 to the housing 20. In turn, pin 29a mounts to the housing 20.

Each upper flange, e.g., flange 45a, also includes a force-actuating flange or drive flange 47 which is aligned along longitudinal axis “A” and which abut the drive assembly 150 such that pivotal movement of the handle 40 forces actuating flange against the drive assembly 150 which, in turn, closes the jaw members 110 and 120. As the handle 40 is squeezed and flange 76′ is incorporated into fixed handle 50, the driving flange 47, through the mechanical advantage of the above-the-center pivot points, biases flange 154 of drive ring 159 which, in turn, compresses a spring 67 against a rear ring 156 of the drive assembly 150 (FIG. 5B). As a result thereof, the rear ring 156 reciprocates sleeve 65 proximally which, in turn, closes jaw member 110 onto jaw member 120. Using the over-the-center pivoting mechanism enables the user to selectively compress the coil spring 67 a specific distance which, in turn, imparts a specific pulling load on the reciprocating sleeve 65 which is converted to a rotational torque about the jaw pivot pin 103. As a result, a specific closure force can be transmitted to the opposing jaw members 110 and 120.

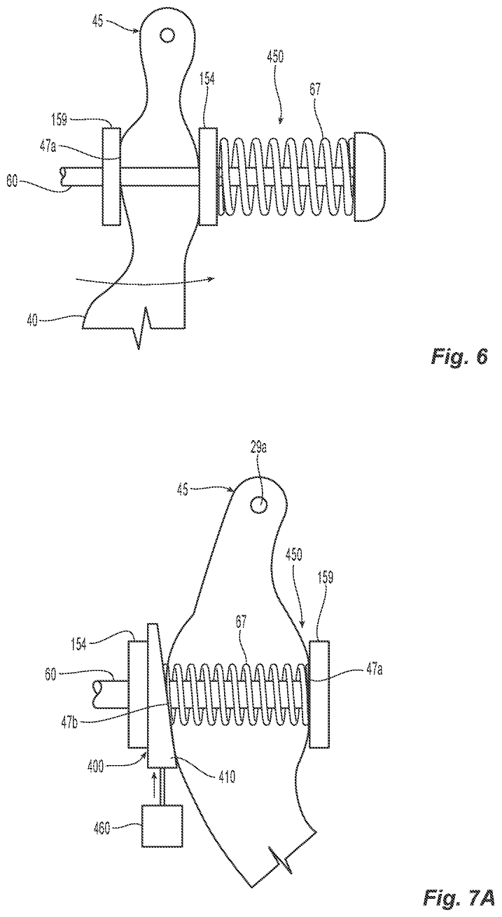

As mentioned above, once the jaw members 110, 120 are closed about tissue under the above-identified closure pressure and the jaw members 110, 120 are energized to seal tissue, any forces associated with tissue expansion are offset by the additional closure pressure associated with the various anti-backdrive assemblies described herein. FIG. 6 is a schematic representation of a drive assembly 450 that may be utilized with one or more of the anti-backdrive assemblies discussed below. Drive assembly 450 is similar to the drive assembly 150 discussed above and generally includes biasing flange 154, drive ring 159, spring 67 and sleeve 65 which cooperate as described above to actuate the jaw members 110, 120.

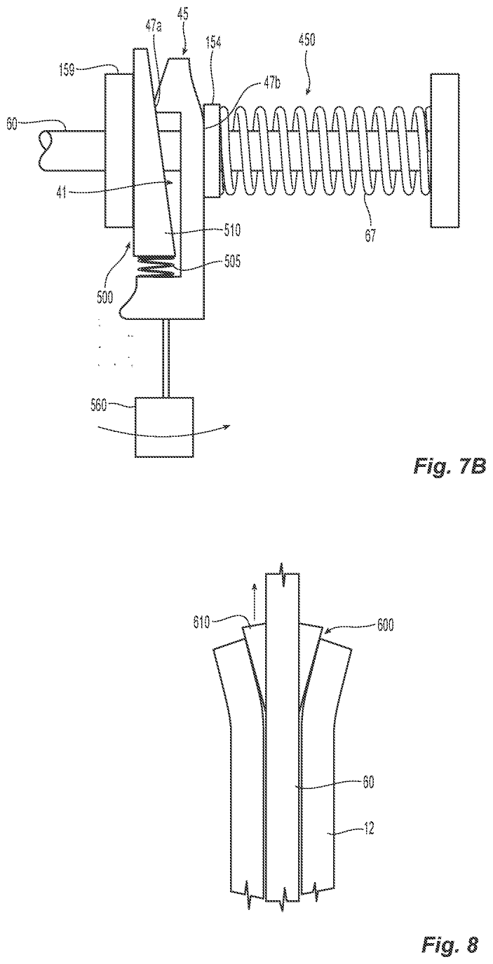

For example, FIG. 7A shows one embodiment of an anti-backdrive assembly 400 for use with the drive assembly 450. Drive assembly 450 in this instance is slightly different than the drive assembly 150 described above but operates in a similar fashion. In this instance, the spring 67 is disposed between the biasing flange 154 and the drive ring 159. Anti-backdrive assembly 400 includes a movable drive wedge 410 disposed between the rear drive flange 47b of handle 40 and the biasing flange 154 of drive assembly 450. A solenoid controller 460 is disposed within the housing 20 and is configured to selectively translate a drive rod 405 operably coupled to the drive wedge 410.

When configured for use with solenoid controller 460, the drive rod 405 is operably coupled to the solenoid controller 460 such that, upon request or in accordance with a sealing algorithm, the solenoid controller 460 provides additional closure force to the jaw members 110, 120 by extending the drive rod 405 which, in turn, moves the drive wedge further between the biasing flange 154 and the drive flange 47b thereby increasing closure pressure. One or more sensors or other types of feedback mechanisms (not shown) may be utilized to communicate with the solenoid controller 460 to regulate the additional closure pressure to ensure the closure pressure continually falls within the above-identified closure pressure range during the entire sealing process. Alternatively, the solenoid controller 460 may be configured to cooperate with a sealing algorithm and regulated accordingly to apply additional closure pressure in accordance therewith.

Solenoid controller 460 may continually monitor the jaw members 110, 120 for feedback and adjust the drive rod 405 accordingly to maintain the appropriate closure pressure between the jaw members 110, 120 during the entire sealing process. Various types of sensors (not shown) or algorithms may be utilized for this purpose. Once sealed, the drive rod 405 is fully retracted to allow the jaw members 110, 120 to open via handle 40 (or 40′).

FIG. 7B shows another embodiment of an anti-backdrive assembly 500 for use with the drive assembly 450. Drive assembly 450 in this instance is similar to drive assembly 150 described above. Anti-backdrive assembly 500 includes a movable drive wedge 510 disposed between drive flange 47a of handle 40 and drive ring 159 of drive assembly 450. A thermally controlled spring 505 is disposed within a cutout 41 defined within handle 40 and is configured to operably couple to the drive wedge 510. A thermal controller 560 is disposed within the housing 20 and is configured to thermally actuate the thermally controlled spring 505 to further wedge the drive wedge 510 between the drive flange 47a and drive ring 159 resulting in additional closure pressure between the jaw members 110, 120. Any type of thermally activated spring is envisioned and may be utilized for this purpose, e.g., compression spring, extension spring, torsion spring, etc.

When configured for use with thermal controller 560, the thermally controlled spring 505, upon request or in accordance with a sealing algorithm, provides additional closure force to the jaw members 110, 120 by forcing the drive wedge between the drive flange 47a and drive ring 159 thereby increasing closure pressure. One or more sensors or other types of feedback mechanisms (not shown) may be utilized to communicate with the thermal controller 560 to regulate the additional closure pressure to ensure the closure pressure continually falls within the above-identified closure pressure range during the entire sealing process. Alternatively, the thermal controller 560 may be configured to cooperate with a sealing algorithm and regulated accordingly to apply additional closure pressure in accordance therewith.

Thermal controller 560 may continually monitor the jaw members 110, 120 for feedback and adjust thermal response of the thermally controlled spring 505 accordingly to maintain the appropriate closure pressure between the jaw members 110, 120 during the entire sealing process. Various types of sensors (not shown) or algorithms may be utilized for this purpose. Once sealed, thermally controlled spring 505 is fully retracted by regulating the temperature thereof to allow the jaw members 110, 120 to open via handle 40 (or 40′).

FIG. 8 shows another embodiment of an anti-backdrive assembly 600 for use with forceps 10, 10′. Unlike the aforedescribed anti-backdrive assemblies 400, 500 discussed above, anti-backdrive assembly 600 cooperates with drive sleeve 65 to limit recoil of the drive sleeve 65 during tissue expansion. More particularly, anti-backdrive assembly 600 includes a drive ferrule 610 disposed between the outer shaft 12 and the drive sleeve 60. Drive ferrule 610 is operably coupled to the handle 40 such that upon actuation of the drive assembly 450, drive sleeve 65 is translated to close the jaw members 110, 120 as described above. At the same time or at the end of the actuation stroke of handle 40, the drive ferule 610 is forced between the shaft 12 and the drive sleeve 65. As such, the drive sleeve 65 is locked against the shaft 12 and is configured to prevent recoil return (i.e., which may be extension or retraction depending upon the forceps 10).

As mentioned above, during sealing, tissue expansion forces may be large enough to pry the jaw members 110, 120 away from one another and reduce the closure pressure therebetween. Drive ferrule 610 prevents the drive sleeve 65 from recoiling thereby counteracting any increase in closure pressures during tissue expansion. Anti-backdrive assembly 600 acts more passively than the other aforementioned anti-backdrive assemblies 400, 500 and only when the expansion forces associated with tissue sealing cause the closure pressure between jaw members 110, 120 to fall will anti-backdrive 600 work to counteract these forces to maintain the closure pressure within the appropriate closure pressure range. Upon release of the handle 40, the drive ferrule 610 is retracted to allow translation of the drive sleeve 65.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A vessel sealing instrument, comprising:
a housing having a shaft extending from a distal end thereof, a distal end of the shaft having an end effector assembly including a pair of opposing first and second jaw members operably coupled thereto;
a drive assembly disposed within the housing and configured to move the jaw members upon actuation thereof between an open position and a closed position for clamping tissue with a closure pressure within the range of about 3 kg/cm² to about 16 kg/cm²;
an anti-backdrive assembly operably disposed within the housing and including a drive wedge; and
a solenoid controller operably coupled to the drive wedge and configured to selectively move the drive wedge into the drive assembly upon activation thereof to increase the closure pressure between the jaw members in response to tissue expansion during sealing.

2. The vessel sealing instrument according to claim 1, wherein the solenoid controller selectively moves the drive wedge in response to a sealing algorithm.

3. The vessel sealing instrument according to claim 1, wherein the solenoid controller selectively moves the drive wedge in response to a sensor.

4. The vessel sealing instrument according to claim 1, wherein the housing includes a moveable handle disposed therein operably coupled to the drive assembly, actuation of the moveable handle correspondingly moves the drive assembly to move the jaw members between open and closed positions.

5. The vessel sealing instrument according to claim 4, wherein at least a portion of the handle is disposed between a drive ring and a biasing flange of the drive assembly, actuation of the handle biases the biasing flange and the drive ring relative to one another to move the jaw members.

6. The vessel sealing instrument according to claim 5, wherein the drive wedge is disposed between the handle and the drive ring or the handle and the biasing flange such that upon actuation of the solenoid controller, the drive wedge further biases the drive ring and the biasing flange relative to one another.

7. The vessel sealing instrument according to claim 1 wherein the drive assembly includes a spring configured to regulate the closure pressure between the jaw members.

8. A vessel sealing instrument, comprising:
a housing having a shaft extending from a distal end thereof, a distal end of the shaft having an end effector assembly including a pair of opposing first and second jaw members operably coupled thereto;
a drive assembly disposed within the housing and configured to move the jaw members upon actuation thereof between an open position and a closed position for clamping tissue with a closure pressure within the range of about 3 kg/cm² to about 16 kg/cm²;
an anti-backdrive assembly operably disposed within the housing and including a drive wedge; and
a thermally controlled spring operably coupled to the drive wedge, the thermally controlled spring transitional between a first configuration and at least one second configuration upon activation thereof via a change in temperature, the thermally controlled spring configured to selectively move the drive wedge into the drive assembly upon transition thereof to increase the closure pressure between the jaw members in response to tissue expansion during sealing.

9. The vessel sealing instrument according to claim 8, wherein the thermally controlled spring is operably coupled to a thermal controller configured to selectively transition the thermally controlled spring.

10. The vessel sealing instrument according to claim 9, wherein the thermal controller selectively moves the thermally controlled spring and drive wedge in response to a sealing algorithm.

11. The vessel sealing instrument according to claim 9, wherein the thermal controller selectively moves the thermally controlled spring and drive wedge in response to a sensor.

12. The vessel sealing instrument according to claim 8, wherein the housing includes a moveable handle disposed therein operably coupled to the drive assembly, actuation of the moveable handle correspondingly moves the drive assembly to move the jaw members between open and closed positions.

13. The vessel sealing instrument according to claim 12, wherein at least a portion of the handle is disposed between a drive ring and a biasing flange of the drive assembly, actuation of the handle biases the biasing flange and the drive ring relative to one another to move the jaw members.

14. The vessel sealing instrument according to claim 13, wherein the drive wedge is disposed between the handle and the drive ring or the handle and the biasing flange such that upon actuation of the thermal controller and thermally controlled spring, the drive wedge further biases the drive ring and the biasing flange relative to one another.

15. The vessel sealing instrument according to claim 8, wherein the drive assembly includes a spring configured to regulate the closure pressure between the jaw members.

16. The vessel sealing instrument according to claim 8, wherein the drive wedge and thermally controlled spring are disposed within a cutout defined within the handle.
